(19) United States
(12) Patent Application Publication
(10) Pub. No.: US 2004/0247692 A1
(43) Pub. Date: Dec. 9, 2004

(54) Method for fabricating aspherical lens

(76) Inventor: Ga-lane Chen, Fremont, CA (US)

Correspondence Address: WEI TE CHUNG, FOXCONN INTERNATIONAL, INC., 1650 MEMOREX DRIVE, SANTA CLARA, CA, 95050, US

(21) Appl. No.: 10/861,020

(22) Filed: Jun. 3, 2004

(30) Foreign Application Priority Data
Jun. 3, 2003 (TW) 92115119

(51) Int. Cl. B29D 11/00; A61K 9/22; A61K 9/50; G02B 27/10

(52) U.S. Cl. 424/501; 424/468

(57) ABSTRACT

A method for fabricating an aspherical lens includes the steps of: providing an aspherical lens (10, 10′) having an aspherical surface (101, 101′) and an opposite flat surface (102, 102′); and defining a multiple-step grating in the flat surface by a photolithographic method. The aspherical lens has a reduced chromatic aberration effect, and can thus provide a clear image. Furthermore, an effect of the aspherical lens having the grating is equivalent to that of a conventional aspherical lens unit. Thus when the aspherical lens is used in an optical system, it can reduce a bulk and a weight of the optical system.

### BACKGROUND OF THE INVENTION

1. Field of the Invention

This invention relates generally to methods for fabricating optical elements, and more particularly to methods for fabricating aspherical lenses.

2. Description of the Related Art

Aspherical lenses are widely used in aspherical lens systems of vidicons, cameras and so on. U.S. Pat. Nos. 5,861,999 discloses a conventional aspherical lens system. The aspherical lens system comprises a plurality of aspherical lens units. Each aspherical lens unit comprises two aspherical lenses with a clearance defined therebetween. A chromatic aberration of the aspherical lens unit can be adjusted by adjusting the clearance. U.S. Pat. Nos. 5,557,472 and 5,513,045 also disclose such conventional aspherical lens systems.

Optical characteristics of each aspherical lens unit are determined in large part by the physical geometry of each of the aspherical lenses thereof. If either aspherical lens is imprecisely formed, this can result in an unacceptably high chromatic aberration effect. Furthermore, the aspherical lens system comprising a plurality of aspherical lens units is relatively bulky and heavy, and unsuited for miniaturized applications.

Therefore, a method for fabricating an improved aspherical lens that overcomes the above-described problems is desired.

### BRIEF SUMMARY OF THE INVENTION

Accordingly, an object of the present invention is to provide a method for fabricating an aspherical lens such that: (1) the aspherical lens has a reduced chromatic aberration effect; and (ii) when the aspherical lens is used in an aspherical lens system, it can reduce a bulk and a weight of the aspherical lens system.

To achieve the above-mentioned object, a method for fabricating an aspherical lens comprises the steps of: providing an aspherical lens having an aspherical surface and an opposite flat surface; and defining a multiple-step grating in the flat surface by a photolithographic method.

The aspherical lens fabricated by the method of the present invention can reduce the chromatic aberration effect thereof by adjusting phases thereof by using the grating. Thus, the aspherical lens can provide a clear image. Furthermore, an effect of the aspherical lens having the grating is equivalent to that of a conventional aspherical lens unit. Thus, when the aspherical lens having the grating is used in an aspherical lens system, it can reduce a bulk and a weight of the aspherical lens system.

Other objects, advantages and novel features of the invention will become more apparent from the following detailed description when taken in conjunction with the accompanying drawings, in which:

### BRIEF DESCRIPTION OF THE DRAWINGS

FIG. 1 is a schematic side elevation of an aspherical lens in accordance with the present invention;

FIG. 2 is a schematic side cross-sectional view of a first mask used in a first optical lithography process of the present invention;

FIG. 3 is similar to FIG. 1, but showing a first photoresist film formed on the aspherical lens;

FIG. 4 is similar to FIG. 3, but showing the first mask placed on the first photoresist film and a first exposure process being performed;

FIG. 5 is similar to FIG. 4, but showing the first photoresist film changed after a first developing process has been performed;

FIG. 6 is similar to FIG. 5, but showing the aspherical lens changed after a first etching process has been performed;

FIG. 7 is similar to FIG. 6, but showing the aspherical lens after residual portions of the first photoresist film has been removed;

FIG. 8 is a schematic side cross-sectional view of a second mask used in a second optical lithography process of a first embodiment of the present invention;

FIG. 9 is similar to FIG. 7, but showing a second photo resist film formed on the aspherical lens;

FIG. 10 is similar to FIG. 9, but showing the second mask placed on the second photoresist film and a second exposure process being performed in accordance with the first embodiment of the present invention;

FIG. 11 is similar to FIG. 10, but showing the second photoresist film changed after a second developing process has been performed;

FIG. 12 is similar to FIG. 11, but showing the aspherical lens changed after a second etching process has been performed;

FIG. 13 is similar to FIG. 12, but showing the completed aspherical lens after residual portions of the second photo resist film have been removed;

FIG. 14 is a schematic side elevation of a third mask used in a second optical lithography process of a second embodiment of the present invention;

FIG. 15 is similar to FIG. 9, except that the photo resist film formed on the aspherical lens is defined as a third photo resist film in accordance with the second embodiment of the present invention;

FIG. 16 is similar to FIG. 15, but showing the third mask placed on the third photoresist film and a second exposure process being performed in accordance with the second embodiment of the present invention;

FIG. 17 is similar to FIG. 16, but showing the third photoresist film changed after a second developing process has been performed;

FIG. 18 is similar to FIG. 17, but showing the aspherical lens changed after a second etching process has been performed; and

FIG. 19 is similar to FIG. 18, but showing the completed aspherical lens after residual portions of the third photoresist film have been removed.

### DESCRIPTION OF THE PREFERRED EMBODIMENTS

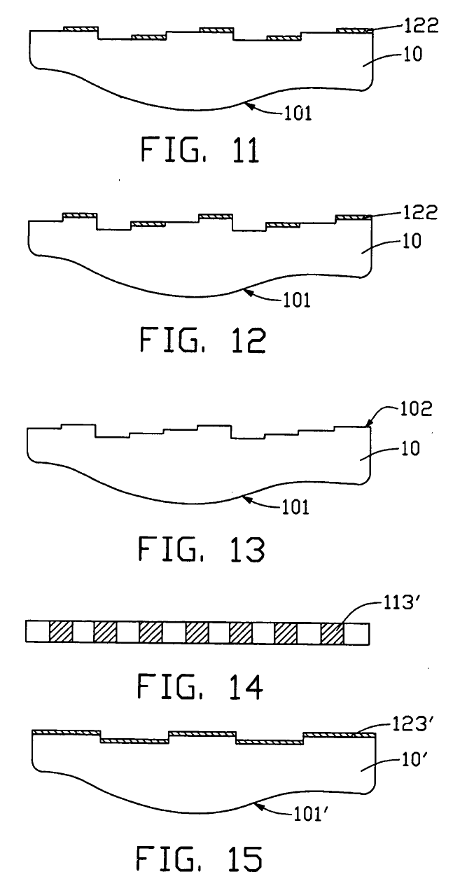

FIGS. 1 to 13 show a first embodiment of a method of the present invention. The first embodiment of the method comprises the following steps:

(a) providing an aspherical lens 10; and (b) defining a multiple-step grating in the aspherical lens 10.

Referring to FIG. 1, a schematic side elevation of the aspherical lens 10 is shown. The aspherical lens 10 has an aspherical surface 101 and an opposite flat surface 102. The flat surface 102 is formed by a mechanical polishing process or a chemical polishing process.

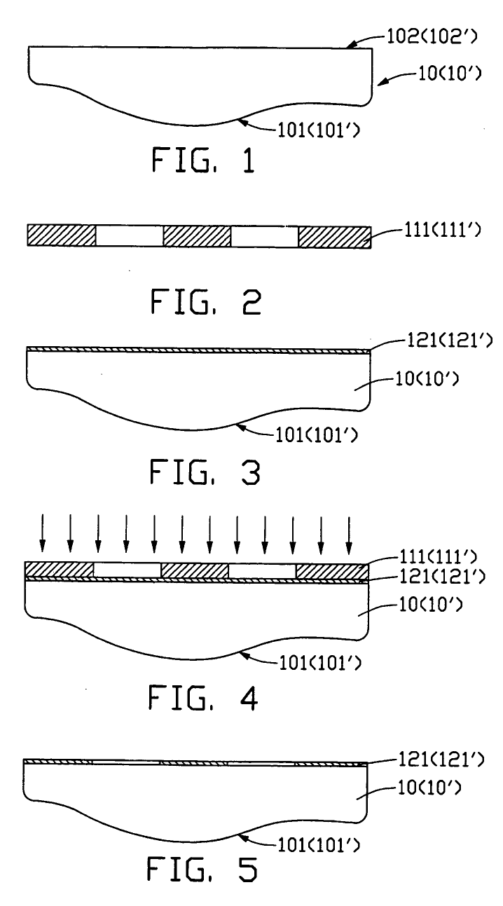

FIGS. 2 to 13 show successive stages of step (b). Firstly, a first photoresist film 121 is formed on the flat surface 102, as shown in FIG. 3. Secondly, a first mask 111 (shown in FIG. 2) is placed on the first photoresist film 121. A size of the first mask 111 is the same as that of the flat surface 102, and a resolution of the first mask 111 is 100 lines/mm.

Thirdly, a grating with a set of steps is formed in the flat surface 102 by first exposure, developing and etching processes. FIG. 4 is a schematic side cross-sectional view showing a first exposure process being performed on the aspherical lens 10 having the first mask 111 placed thereon. The first mask 111 is placed on the first photoresist film 121, and preferably an ultraviolet radiation exposure process is executed by an aligner or a stepper. Alternatively, an exposure process can be executed by an electron beam writer.

Then the photoresist portions of the first photoresist film 121 corresponding to the exposed areas are developed (see FIG. 5).

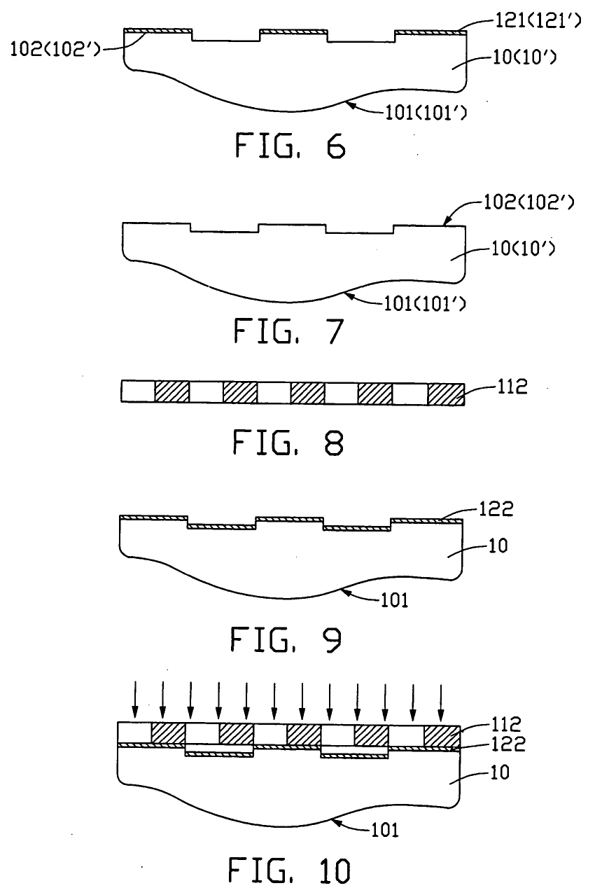

FIG. 6 shows a first etching process being performed on the aspherical lens 10 after the first developing process has been completed. The first etching process is executed by a photo etching technology or a μ-ray developing technology. The first etching process is controlled by a computer, so that it is performed only on those portions of the flat surface 102 that have been developed. Thus, a plurality of recesses is produced, with a depth of the recesses being determined by the steps desired to be in the fully completed grating.

Referring to FIG. 7, the residual portions of the first photoresist film 121 are removed by eroding the surface 102 using a potassium cyanide solution, or by using another suitable technique. Thus, a precursor grating with a set of double steps is formed on the surface 102.

Referring to FIG. 8, a second mask 112 used for producing a multi-step grating is shown. A resolution of the second mask 112 is 200 lines/mm. Referring to FIGS. 9-12, a second photo resist film 122 is formed on the surface 102 of the aspherical lens 10, and the second mask 112 is placed on the second photo resist film 122. Second exposure, developing and etching processes are respectively performed, in similar fashion to the first exposure, developing and etching processes described above. A depth of recesses produced by the second etching process is half of that of the recesses produced by the first etching process. Finally, residual portions of the second photo resist film 122 are removed by eroding the surface 102 using a potassium cyanide solution, or by using another suitable technique. Thus, an aspherical lens 10 having a grating with a plurality of nonsymmetrical steps formed thereon is obtained.

By essentially repeating the above-described steps “n” times, each time using a mask having a resolution of (2ⁿ)*(R) (R denoting a resolution of the first mask 111, and n being a positive integer), and by controlling a depth of the recesses produced in each etching process to be half that of the recesses produced in the respective immediately preceding etching process, a 2ⁿ⁺¹-step grating can be formed.

A second embodiment of the method of the present invention is as follows. Referring to FIGS. 1-7, according to the first embodiment, a grating with a set of double steps is formed on the surface 102′.

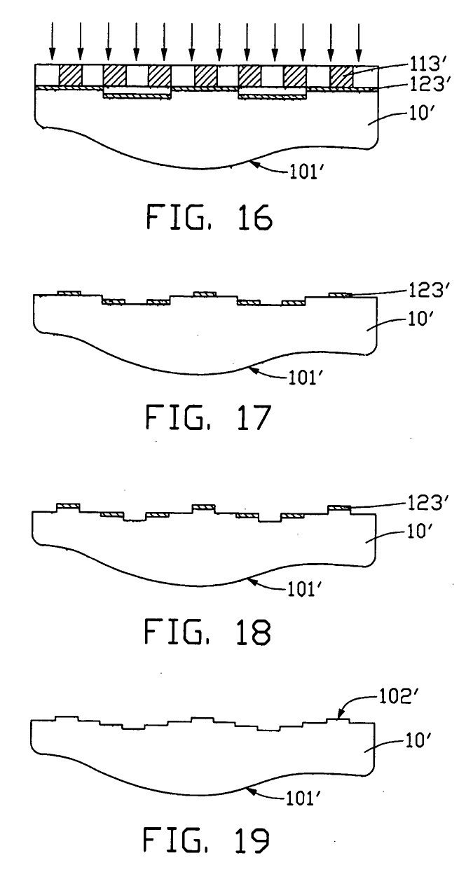

Referring to FIG. 14, a third mask 113′ used for producing a multi-step grating is shown. A resolution of the third mask 113′ is 300 lines/mm. Then, referring to FIGS. 15-18, a third photo resist film 123′ is formed on the surface 102′ of the aspherical lens 10′, and second exposure, developing and etching processes are respectively performed. A depth of recesses produced in the second etching process is half that of the recesses produced in the first etching process. Finally, residual portions of the third photo resist film 123′ are removed by eroding the surface 102′ using a potassium cyanide solution, or by using another suitable technique. Thus, an aspherical lens 10′ having a grating with a plurality of symmetrical steps formed thereon is obtained.

It is understood that the above-described embodiments are intended to illustrate rather than limit the invention. Variations may be made to the embodiments without departing from the spirit of the invention. Accordingly, it is appropriate that the appended claims be construed broadly and in a manner consistent with the scope of the invention.

What is claimed is:

1. A method for fabricating an aspherical lens, comprising the steps of:
(a) providing an aspherical lens, the aspherical lens having an aspherical surface and an opposite flat surface; and
(b) defining a multiple-step grating in the flat surface.

2. The method as claimed in claim 1, wherein step (b) comprises the steps of:
(b1) forming a photoresist film on the flat surface;
(b2) placing a mask on the photoresist film, a resolution of the mask being R;
(b3) forming a grating with a set of steps in the flat surface by first exposure, developing and etching processes; and
(b4) repeating the exposure, developing and etching processes n times, each time using a mask having a resolution of (2ⁿ)*(R), wherein n is a positive integer, whereby a corresponding 2ⁿ⁺¹-step grating is obtained.

3. The method as claimed in claim 2, wherein each exposure process is an ultraviolet radiation exposure process executed by an aligner or a stepper.

4. The method as claimed in claim 2, wherein each exposure process is executed by an electron beam writer.

5. The method as claimed in claim 2, wherein each etching process is executed by using a photo etching technology or a μ-ray developing technology.

6. The method as claimed in claim 2, wherein a depth of recesses produced in each etching process is approximately half that of recesses produced in an immediately preceding etching process.

7. The method as claimed in claim 2, wherein R is approximately 100 lines/mm.

8. The aspherical lens made from the method as claimed in claim 1.

9. A method for fabricating an aspherical lens, comprising the steps of:
(a) providing an aspherical lens, the aspherical lens having an aspherical surface and an opposite flat surface; and
(b) defining a symmetrical multiple-step grating in the flat surface.

10. The method as claimed in claim 9, wherein step (b) comprises the steps of:
(b1) forming a photo resist film on the flat surface;
(b2) placing a mask on the photo resist film, a resolution of the mask being R;
(b3) forming a grating with a set of steps in the flat surface by first exposure, developing and etching processes; and
(b4) repeating the exposure, developing and etching processes once, using a mask having a resolution of 3R, whereby a corresponding 4-step grating is obtained.

11. The method as claimed in claim 10, wherein each exposure process is an ultraviolet radiation exposure process executed by an aligner or a stepper.

12. The method as claimed in claim 10, wherein each exposure process is executed by an electron beam writer.

13. The method as claimed in claim 10, wherein each etching process is executed by using a photo etching technology or a μ-ray developing technology.

14. The method as claimed in claim 10, wherein a depth of recesses produced in the repeated etching process is approximately half that of recesses produced in the first etching process.

15. The method as claimed in claim 10, wherein R is approximately 100 lines/mm.

16. The aspherical lens made from the method as claimed in claim 9.

17. A method of making a lens comprising steps of:
a. providing a lens including a curved surface and a flat surface opposite to each other;
b. forming a multi-step configuration on the flat surface in cycle.

18. The method as claimed in claim 17, wherein step (b) includes:
b1. applying a patterned photoresist film on the flat surface with some specific pattern exposing some portions of the lens without protection of the photoresist film;
b2 etching said some portions;
b3. removing the patterned photoresisit film and forming corresponding multi-step thereof accordingly.

19. The method as claimed in claim 18, wherein said step (b1) includes:
b11. applying a full un-patterned photoresisit film on the flat surface;
b12. applying a mask on the photoresisit film wherein said mask defines said specific pattern; and
b13. removing portions of the photoresist film via an exposure process according to said specific pattern.

20. The method as claimed in claim 17, wherein step (b) is repeated under a condition that the patterns of the photoresisit films become finer and finer so as to form more finer multi-step configurations thereof accordingly.
